(19) United States
(12) Patent Application Publication
(10) Pub. No.: US 2026/0165562 A1
(43) Pub. Date: Jun. 18, 2026

(54) GUIDEWIRE TIP WITH COILED MEMBER

(71) Applicant: BOSTON SCIENTIFIC SCIMED, INC., Maple Grove, MN (US)

(72) Inventors: Jessica Rincon Nieto, Boston, MA (US); Laura Emily Sabban, Norton, MA (US); Murillo Murillo Sebastian, San Jose (CR)

(73) Assignee: BOSTON SCIENTIFIC SCIMED, INC., Maple Grove, MN (US)

(21) Appl. No.: 19/423,485

(22) Filed: Dec. 17, 2025

Related U.S. Application Data
(60) Provisional application No. 63/735,507, filed on Dec. 18, 2024.

(51) Int. Cl. A61B 1/00

(52) U.S. Cl. CPC A61B 1/00154

(57) ABSTRACT

A guidewire includes a core wire having a proximal end, a distal end, and a tapered portion therebetween. A coil disposed about a distal portion of the core wire, the coil having a proximal portion, a distal portion, and a central portion therebetween, wherein the proximal and distal portions have a first pitch, and the central portion has a second pitch. A first outer coating comprising a first polymeric material extending distally along a length of the core wire from the proximal end to the tapered portion, and a second outer coating comprising a second polymeric material extending proximally along a length of the core wire from the distal end to the tapered portion.

### CROSS REFERENCE TO RELATED APPLICATIONS

The present application claims the benefit of U.S. Provisional Patent Application Ser. No. 63/735,507, filed Dec. 18, 2024, the disclosure of which is incorporated herein by reference.

### TECHNICAL FIELD

The disclosure pertains to guidewires with an improved distal tip. More particularly, the present invention pertains to guidewires for use in endoscopic retrograde cholangiopancreatography.

### BACKGROUND

A wide variety of medical devices have been developed for medical use including, for example, endoscopic guidewires for use in navigating biliary ducts. Because the pancreaticobiliary ducts may be tortuous, it is desirable to combine a number of performance features in a guidewire. For example, it may be desirous to provide a guidewire with increased rail support, variable stiffness, resistance to kinking, and increased tactile feedback. A number of different guidewire structures and assemblies are known, however there is an ongoing need to provide alternative guidewire structures and assemblies.

### SUMMARY

This disclosure provides design, material, manufacturing method, and use alternatives for medical devices. An example medical device includes a guidewire comprising a core wire having a proximal end, a distal end, and a tapered portion therebetween, a coil disposed about a distal portion of the core wire, the coil having a proximal portion with a first pitch, a distal portion with a second pitch, and a central portion therebetween having a third pitch, a first outer coating comprising a first polymeric material, the first coating extending distally along a length of the core wire from the proximal end to the tapered portion, a second outer coating comprising a second polymeric material extending proximally along a length of the core wire from the distal end to the tapered portion, wherein a proximal end of the second outer coating is configured to abut a distal end of the first outer coating, and wherein the core wire has a first diameter proximal of the tapered portion, and a second diameter distal of the tapered portion, the first diameter greater than the second diameter.

Alternatively or additionally to the embodiment above, the core wire comprises a nitinol wire.

Alternatively or additionally to any of the embodiments above, the coil is a nitinol spring coil.

Alternatively or additionally to any of the embodiments above, the coil is a tungsten spring coil.

Alternatively or additionally to any of the embodiments above, the second coating is doped with a radiopaque material.

Alternatively or additionally to any of the embodiments above, the second coating extends past a distal end of the core wire to form an atraumatic tip.

Alternatively or additionally to any of the embodiments above, the proximal portion of the coil is disposed around the tapered portion of the core wire.

Alternatively or additionally to any of the embodiments above, the proximal portion of the coil is disposed around the core wire distal of the tapered portion.

Alternatively or additionally to any of the embodiments above, the coil extends distally beyond the distal end of the core wire.

Alternatively or additionally to any of the embodiments above, the coil has an inside diameter substantially equal to an outside diameter of the core wire.

Alternatively or additionally to any of the embodiments above, the first and second pitches are smaller than the third pitch.

Alternatively or additionally to any of the embodiments above, the central portion is an area of increased flexibility relative to the proximal and distal portions of the coil.

Alternatively or additionally to any of the embodiments above, the guidewire is configured to bend at the area of increased flexibility.

Alternatively or additionally to any of the embodiments above, the proximal end of the coil is welded to the core wire.

Alternatively or additionally to any of the embodiments above, the central portion of the coil is located 2-4 centimeters from the distal end of the core wire.

An example medical device includes a guidewire comprising a core wire having a proximal end, a distal end, and a tapered portion therebetween, the core wire comprising nitinol wire, a coil disposed about a distal portion of the core wire, the coil having a proximal portion with a first pitch, a distal portion with a second pitch, and a central portion therebetween having a third pitch, wherein the third pitch is greater than the first and second pitches, a first outer coating comprising a first polymeric material extending distally along a length of the core wire from the proximal end to the tapered portion, a second outer coating comprising a second polymeric material extending proximally along a length of the core wire from the distal end to the tapered portion, wherein a proximal end of the second outer coating is configured to abut a distal end of the first outer coating, and wherein the core wire has a first diameter proximal of the tapered portion, and a second diameter distal of the tapered portion, the first diameter greater than the second diameter.

Alternatively or additionally to the embodiment above, the coil is a nitinol spring coil.

Alternatively or additionally to any of the embodiments above, the coil is a tungsten spring coil.

Alternatively or additionally to any of the embodiments above, the second coating is doped with a radiopaque material.

Alternatively or additionally to any of the embodiments above, the second coating extends past a distal end of the core wire to form an atraumatic tip.

Alternatively or additionally to any of the embodiments above, the proximal portion of the coil is disposed around the tapered portion of the core wire.

An example medical device includes a guidewire comprising a core wire having a proximal end, and a distal tapered portion, a coil disposed about a distal portion of the core wire, the coil having a proximal portion with a first pitch, a distal portion with a second pitch, and a central portion therebetween having a third pitch, a first outer coating comprising polytetrafluoroethylene extending distally along a length of the core wire from the proximal end to the tapered portion, a second outer coating comprising aliphatic polyether polyurethane extending proximally along a length of the coil from the distal portion to the proximal portion, wherein a proximal end of the second outer coating is configured to abut a distal end of the first outer coating.

Alternatively or additionally to the embodiment above, the third pitch is greater than the first and second pitches, and is configured to form an area of increased flexibility relative to the first and second pitches.

Alternatively or additionally to any of the embodiments above, the guidewire is configured to bend at the area of increased flexibility.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a cross-sectional view of an example distal end of a guidewire;

FIG. 2 is a cross-sectional view of the distal end of the guidewire of FIG. 1;

FIG. 3 is a cross-sectional view of the distal end of another example guidewire;

FIG. 4 is a cross-sectional view of the distal end of another example guidewire.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about,” in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

During endoscopic retrograde cholangiopancreatography (ERCP) procedures, endoscopic guidewires facilitate access to the pancreaticobiliary ducts, enabling the passage and exchange of instruments for diagnosis and therapeutic interventions. Traditional guidewires face significant limitations, specifically navigation challenges when maneuvering through complex duct anatomy, and lack of support for advancing therapeutic devices smoothly. For example, a stiffer guidewire may be better suited for device exchange to increase rail support, cannulation, and stent placement, while a more flexible guidewire may be needed for navigation within ducts. These limitations can prolong procedures and complicate treatment, increasing the risk of biliary perforation, and lead to difficulty positioning medical devices.

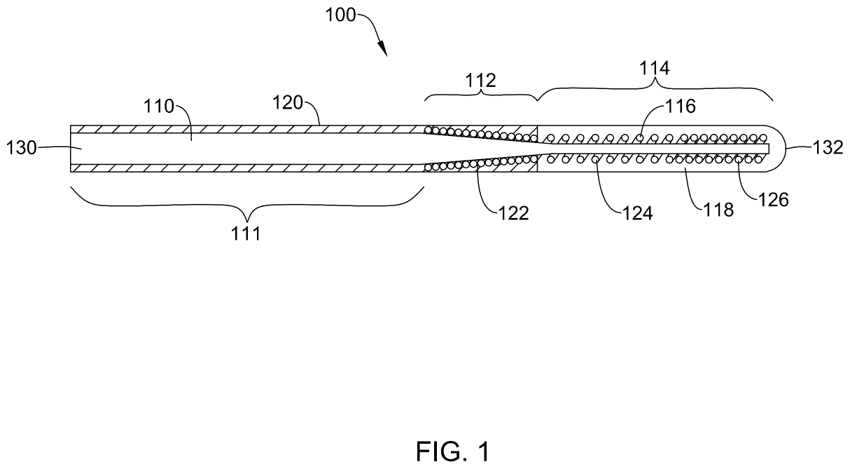

These challenges require a guidewire to have variable stiffness as described in this disclosure. For example, FIG. 1 is an example embodiment of a distal end region of a guidewire 100. The guidewire 100 includes a proximal end 130, a distal end 132, and a core wire 110. The core wire 110 can be made of any suitable material including metal, metal alloys (e.g., nitinol), polymers or mixtures thereof. The core wire may have a preformed shape, and includes a proximal portion 111, a distal portion 114, and a tapered portion 112 therebetween. The proximal portion 111 may have a diameter greater than the diameter of the distal portion 114, with the tapered portion 112 forming a continuous, gradual transition between the areas of varying diameter. In some embodiments the core wire 110 may include a plurality of distal segments with decreasing diameter, or the core wire 110 may have a single, generally tapered distal end. It should be understood that a vast number of configurations of segments and tapered portions may be included without departing from the scope of the disclosure.

The guidewire 100 has a first coating 120 disposed around a proximal portion of the core wire 110, and a second coating 118 disposed around a distal portion of the core wire 110. The first coating 120 may be formed of a first polymeric material, such as polytetrafluoroethylene (PTFE) or other suitable materials. The second coating 118 may be formed of a second polymeric material, such as an aliphatic polyether polyurethane (e.g., Tecoflex™), another polymer, or other suitable materials. The first polymeric material may be different from the second polymeric material. One or both of the coatings 118, 120 may be heat shrunk over the core wire 110, dip coated over the core wire 110, spray coated on the core wire 110, or attached by any suitable means. A distal end of the first coating 120 and a proximal end of the second coating 118 may abut one another to form a joint, fully enclosing the core wire 110. The coatings may be disposed over any portion of the core wire 110, for example, the first coating 120 may extend to the tapered region of the core wire 112. In some embodiments the first coating 120 may end proximal of the tapered region 112, distal of the tapered region 112, or along the tapered region 112, for example.

In some embodiments the first coating 120 may include a flared portion configured to receive the second coating 118 to form a joint. In some embodiments, the second coating may be heated or melted to flow under the flared portion of the first coating 120, to form an overlapping joint between the first coating 120 and the second coating 118. The second coating 118 may extend to the distal end of the guidewire 132 to form an atraumatic tip, the tip configured to reduce the risk of perforation. In some embodiments, the first and/or second coatings may be doped with radiopaque materials, or otherwise include a radiopaque material, to aid in determining the location of the guidewire during navigation. Some embodiments may include other radiopaque markers along the length of the guidewire 100.

A spring coil 116 is disposed around a distal portion of the core wire 110. The coil 116 may be comprised of a number of suitable materials, for example tungsten, nitinol, or platinum. In some embodiments, the coil 116 will serve as a radiopaque marker. The coil 116 may be one continuous coiled wire or may be a series of coiled segments. The coil 116 may have areas of variable pitch along the length of the coil 116. As illustrated in FIG. 1, a proximal portion 122 of the coil 116 is disposed around the tapered portion of the core wire 112 and has a small pitch. The proximal portion 122 of the coil 1116 may be tightly wound, in which each successive coil windings of the coil 116 in this proximal portion 122 is touching the adjacent coil windings. Said differently, the pitch of the coil 1116 throughout the proximal portion 122 may be equivalent to the diameter of the wire in the proximal portion 122. Similarly, the distal portion 126 of the coil 116 is tightly wound, and has a small pitch. The small pitch throughout the proximal portion 122 and/or the distal portion 126 (122, 126) may be any suitable distance for example 0.002 to 0.01 inches. These areas of tightly wound coil provide higher rail support, torque control, and more tactile feedback. A central or intermediate portion 124 of the spring coil 116, has a longer pitch greater than the small pitch of the proximal portion 122 and/or distal portion 126, providing flexibility. The length of this intermediate portion 124 of longer pitch may be any suitable distance, for example, 0.004 to 0.05 inches. In some embodiments the pitch is determined by the diameter or thickness of the wire, for example, the pitch throughout the intermediate portion 124 may be between 2 and 5 times larger than the diameter of the wire. In some instances, the longer pitch of the intermediate portion 124 may be 0.004 to 0.05 inches, for example. This area of flexibility 124 can aid in navigating the guidewire and can serve as a bend point along the tip of the guidewire. Preferably, the force required to bend the distal portion 126 of the coil is greater than the force required to bend the central portion 124. It should be understood that there may be any number of variable pitch segments along the length of the coil, or there may be a plurality of coils with various pitches along the length of the core wire 110. In some embodiments, the diameter or thickness of the wire 116 may vary along the length of the spring coil 116. For example, in areas of small pitch (e.g. proximal portion 122 and/or distal portion 126) may be made up of a wire with a smaller diameter compared to areas with a longer pitch (e.g. intermediate portion 124). Similarly, if the coil 116 is made up of a series of coiled segments, each segment may be made up of a wire with varying diameter. It should be understood that there may be any number of variable diameters of wire along the coil 116, or there may be a plurality of coils with various diameters along the length of the core wire 110.

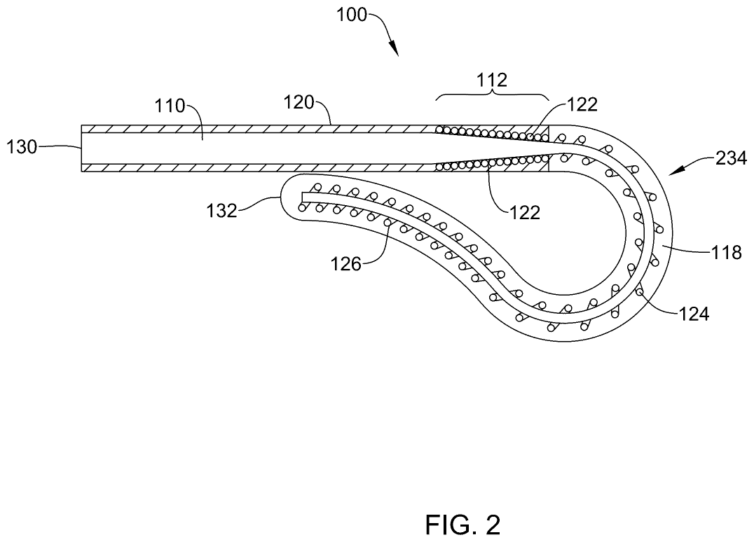

As shown in FIG. 2, the guidewire 100 when in use may bend within a duct, forming a loop. As force is applied to the distal end of the guidewire 100, the distal end 132 moves proximally toward the proximal end 130 of the guidewire 100, and the guidewire 100 curves at the area of increased flexibility 124 to form a bend point 234. The core wire 110 may have a preformed shape such that once force is removed, it will return to its generally straight shape without a bend 234 (FIG. 1). It should be understood that the position and size of the area of increased flexibility 124 may be designed to accommodate a variety of anatomy. More specifically, the size and location of the bend 234 may vary, depending on the location of the variable pitch regions. For example, a shorter central region 124 would result in a smaller loop. Adjusting the position of the central region 124 may cause the guidewire 100 to bend at a different location. For example, a central region 124 close to the distal end 132 would cause the guidewire 100 to bend and loop closer to the distal end 132 and may provide more flexibility for navigation compared to a longer distal coiled portion 126. The location of the flexible coil region 124 may be any suitable distance from the distal tip 132 of the guidewire 100, for example between 2 to 4 centimeters, between 2 to 3 centimeters, or between 3 to 4 centimeters, in some instances. The length of the flexible coil region 124 may be between 0.25 to 7 centimeters, between 1 to 7 centimeters, between 3 to 7 centimeters, between 5 to 7 centimeters, between 0.25 to 5 centimeters, between 0.5 to 2 centimeters, or between 1 to 3 centimeters, in some instances. The proximal and distal coil regions 122 and 126 may be the same length or different lengths. For example, in some embodiments the length of the proximal coil region 122 may be between 5 to 15 centimeters, and the length of the distal coil region 126 may be between 0.25 to 5 centimeters. The coil 116 may be positioned at various locations along the length of the core wire 110. For example, the proximal portion 122 of the coil 116 may be positioned proximal of the tapered portion, distal of the tapered portion, or along the tapered portion of the core wire 110 of the guidewire 100.

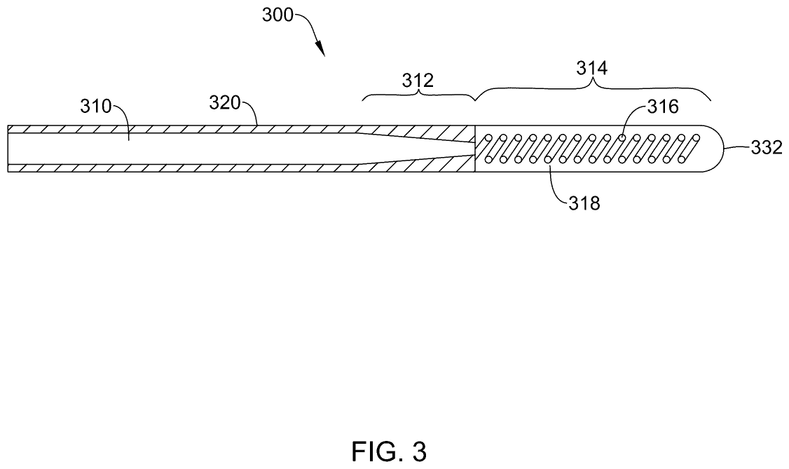

As shown, in FIG. 3, a guidewire 300 has a core wire 310 extending longitudinally through a first coating 320 to reach a second coating 318. A coil 316 abuts the core wire 310 and extends within the second coating 318 toward the distal end of the guidewire. The proximal end of the coil 316 may be welded, bonded, or otherwise secured (e.g., directly) to the distal end of the core wire 310, with the coil 316 extending distally therefrom. The coil 316 extends distally past the distal end of the core wire 310. Accordingly, a majority of the entire length of the lumen through the coil 316 may be devoid of the core wire 310, and in some instances substantially the entire length of the lumen through the coil 316 may be devoid of the core wire 310. The core wire 310 may include a tapered portion 112 or tapered segments as discussed in previous embodiments. Absence of the core wire 310 within the coil 316 allows for a smaller diameter at the distal portion of the guidewire 314, increasing flexibility and torque control at the distal tip 332. The coil 316 may be fixed to the core wire 310 through any suitable method, for example laser or friction welding. Similar to the embodiments described in more detail above, the coil 316 may have areas of variable pitch along the length of the coil 316.

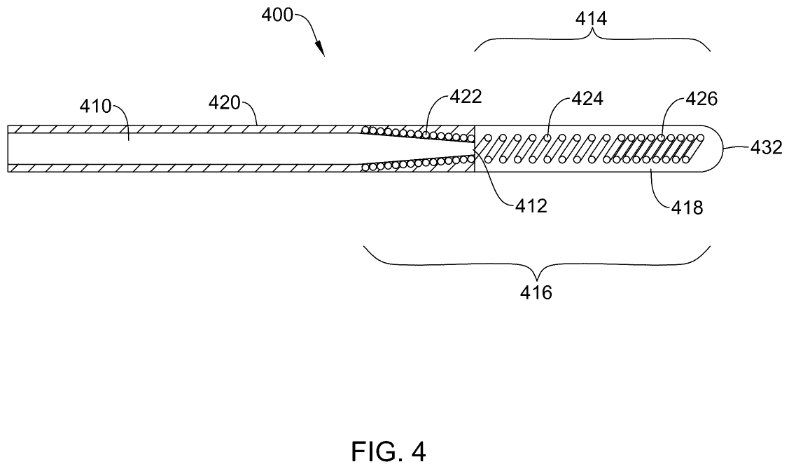

As illustrated in FIG. 4, which is an alternative embodiment of a guidewire 400 made in accordance with the present invention, the guidewire 400 is similar to guidewire 100 except that the core wire 410 does not extend through the length of the coil 416. Accordingly, a majority of the entire length of the lumen through the coil 416 may be devoid of the core wire 410, and in some instances substantially the entire length of the lumen through the coil 416 may be devoid of the core wire 410.

The coil 416 includes variable pitch segments 422, 424 and 426, and extends distally past the core wire 410 toward the distal end of the guidewire 432. The core wire 410 and coil 416 are surrounded by a first coating 420, and a second coating 418. In some embodiments a proximal portion of the coil 416 is disposed around a tapered portion of the core wire 412. In other embodiments, a proximal end of the coil 416 may be fixed to a distal end 412 of the core wire 410. The first coating 420 may surround the core wire 410, while the second coating 418 surrounds the coil 416. In some embodiments, a first portion of the coil 416 may be surrounded by the first coating 420, and a second portion of the coil 416 may be surrounded by the second coating 418. Said differently, the coil 416 may extend from the first coating 420, into the second coating 418. The second coating 418 extends past a distal end of the coil 416 to form an atraumatic tip at a distal end 432 of the guidewire 400.

It will be understood that the dimensions described in association with the above figures are illustrative only, and that other dimensions are contemplated. The materials that can be used for the various components of the guidewire and the various elements thereof disclosed herein may include those commonly associated with medical devices. For simplicity purposes, the following discussion makes reference to the guidewire (and variations, systems or components disclosed herein). However, this is not intended to limit the devices and methods described herein, as the discussion may be applied to other elements, members, components, or devices disclosed herein.

In some embodiments, the guidewire (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL®400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions, or all of the guidewire (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the guidewire (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the guidewire (and variations, systems or components thereof disclosed herein) to achieve the same result.

In some embodiments, the guidewire (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the guidewire (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A guidewire comprising:
an elongate shaft having a proximal end, a distal end, and a tapered portion therebetween;
a coil disposed about a distal portion of the core wire, the coil having a proximal portion with a first pitch, a distal portion with a second pitch, and a central portion therebetween having a third pitch;
a first outer coating comprising a first polymeric material extending distally along a length of the core wire from the proximal end to the tapered portion;
a second outer coating comprising a second polymeric material extending proximally along a length of the core wire from the distal end to the tapered portion;
wherein a proximal end of the second outer coating is configured to abut a distal end of the first outer coating; and
wherein the core wire has a first diameter proximal of the tapered portion, and a second diameter distal of the tapered portion, the first diameter greater than the second diameter.

2. The guidewire of claim 1, wherein the core wire comprises a nitinol wire.

3. The guidewire of claim 1, wherein the coil is a nitinol spring coil.

4. The guidewire of claim 1, wherein the coil is a tungsten spring coil.

5. The guidewire of claim 3, wherein the second coating is doped with a radiopaque material.

6. The guidewire of claim 1, wherein the second coating extends past a distal end of the core wire to form an atraumatic tip.

7. The guidewire of claim 6, wherein the proximal portion of the coil is disposed around the tapered portion of the core wire.

8. The guidewire of claim 6, wherein the proximal portion of the coil is disposed around the core wire distal of the tapered portion.

9. The guidewire of claim 1, wherein the first and second pitches are smaller than the third pitch.

10. The guidewire of claim 9, wherein the second pitch is an area of increased flexibility relative to the first pitch.

11. The guidewire of claim 10, wherein the guidewire is configured to bend at the area of increased flexibility.

12. A guidewire comprising:
a core wire having a proximal end, a distal end, and a tapered portion therebetween, the core wire comprising nitinol wire;
a coil disposed about a distal portion of the core wire, the coil having a proximal portion with a first pitch, a distal portion with a second pitch, and a central portion therebetween having a third pitch, wherein the third pitch is greater than the first and second pitches;
a first outer coating comprising a first polymeric material extending distally along a length of the core wire from the proximal end to the tapered portion;
a second outer coating comprising a second polymeric material extending proximally along a length of the core wire from the distal end to the tapered portion;
wherein a proximal end of the second outer coating is configured to abut a distal end of the first outer coating; and
wherein the core wire has a first diameter proximal of the tapered portion, and a second diameter distal of the tapered portion, the first diameter greater than the second diameter.

13. The guidewire of claim 12, wherein the coil is a nitinol spring coil.

14. The guidewire of claim 12, wherein the coil is a tungsten spring coil.

15. The guidewire of claim 14, wherein the second coating is doped with a radiopaque material.

16. The guidewire of claim 12, wherein the second coating extends past a distal end of the core wire to form an atraumatic tip.

17. The guidewire of claim 6, wherein the proximal portion of the coil is disposed around the tapered portion of the core wire.

18. A guidewire comprising:
a core wire having a proximal end, and a distal tapered portion;
a coil disposed about a distal portion of the core wire, the coil having a proximal portion with a first pitch, a distal portion with a second pitch, and a central portion therebetween having a third pitch;
a first outer coating comprising polytetrafluoroethylene extending distally along a length of the core wire from the proximal end to the tapered portion;
a second outer coating comprising aliphatic polyether polyurethane extending proximally along a length of the coil from the distal portion to the proximal portion;
wherein a proximal end of the second outer coating is configured to abut a distal end of the first outer coating.

19. The guidewire of claim 18, wherein the third pitch is greater than the first and second pitches, and is configured to form an area of increased flexibility relative to the first and second pitches.

20. The guidewire of claim 19, wherein the guidewire is configured to bend at the area of increased flexibility.
